(19) United States
(12) Patent Application Publication
(10) Pub. No.: US 2021/0151131 A1
(43) Pub. Date: May 20, 2021

(54) MULTI-ANALYTE CONCENTRATION ESTIMATION FOR FIXED-WAVELENGTH SPECTROSCOPY

(71) Applicant: Dylan WILKS, New London, NH (US)

(72) Inventor: Dylan WILKS, New London, NH (US)

(21) Appl. No.: 17/098,903

(22) Filed: Nov. 16, 2020

Related U.S. Application Data
(60) Provisional application No. 62/937,368, filed on Nov. 19, 2019.

(51) Int. Cl. G16C 20/40; G16C 20/90; G01N 21/31; G01J 3/42

(52) U.S. Cl. CPC G16C 20/40; G16C 20/90; G01J 2003/283; G01J 3/42; G01N 21/3103

(57) ABSTRACT

The absorbance of a mixed sample at multiple wavelengths is determined and the concentrations of the sample constituents deduced from the observed absorbances. Assuming the sample constituents are known, these wavelengths correspond to peak absorption wavelengths for the constituents. Rather than attempt to generate an analytical relationship among absorbance levels and constituent concentrations, a database of absorbance values for each wavelength, spanning the range of possible analyte concentrations, is employed instead. In general, the wavelengths utilized correspond to peak absorption wavelengths for each of the analytes.

### CROSS-REFERENCE TO RELATED APPLICATION

This application claims priority to, and the benefit of, U.S. Provisional Patent Application No. 62/937,368, filed on Nov. 19, 2019, which is hereby incorporated by reference in its entirety.

### FIELD OF THE INVENTION

The present invention relates to spectroscopic analysis of multiple analytes in a sample.

### BACKGROUND

Spectrometry is used in chemistry to analyze material properties and identity based on the absorbance or transmission—most typically absorbance—of electromagnetic radiation. Analytes in solution typically exhibit wavelength-dependent absorption spectra in which one or more wavelengths of radiation is most strongly absorbed. A spectrophotometer passes radiation of a known wavelength through a sample and records the absorbance. Easily deployed spectrophotometers, which are particularly suited to work in the field, produce radiation at a single but selectable peak wavelength. For a single known analyte, the measured absorbance can be used to estimate the concentration of the analyte in a sample; above a threshold level, concentration varies proportionally with absorbance according to the Beer-Lambert law.

Mixed samples containing more than one analyte of interest can be difficult to analyze with a fixed-output spectrophotometer because each analyte will absorb the incident radiation to some degree, even though one analyte may absorb most strongly. Moreover, the absorbance properties of each analyte in the mixture may differ from that of the pure analyte. Accordingly, there is a need for techniques of analyzing mixed samples based on absorbance levels obtained at multiple wavelengths.

### SUMMARY

In accordance with embodiments of the present invention, a database of measured absorbance values for multiple analytes each at multiple concentration levels and tested with multiple wavelengths is generated. Typically, the wavelengths utilized correspond to peak absorption wavelengths for each of the analytes. A sample is tested at each of the peak wavelengths of the constituent analytes and the resulting absorbance values used to query the database. The concentration combination most closely approximating the obtained readings—i.e., with the least error—is identified. This least-error approach identifies the most likely combination of analyte concentrations despite variation in analyte absorbance characteristics resulting from the mixture.

The approach described herein offers numerous advantages. Generating a database of possible solutions and assessing all or a majority of them helps discriminate among interfering compounds, since the large number of data points limits the deleterious effects on measurement performance that interfering compounds would otherwise impose. For similar reasons, this approach also mitigates the effects of environmental, electrical or other noise sources that would otherwise degrade the quality of the data collected. A least squares or other error value both reveals the existence of conditions that could affect accuracy and indicates the quality of the fit.

Accordingly, in one aspect, the invention pertains to a method of determining concentrations in a sample containing a plurality of known analytes at unknown concentrations, where the sample analytes have different absorption spectra. In various embodiments, the method comprises the steps of providing a database of absorbance values for multiple analytes each at multiple concentration levels and at multiple wavelengths; measuring, with a spectrophotometer, radiation absorptions of the sample at a plurality of wavelengths of incident radiation, the plurality of wavelengths corresponding to peak absorption wavelengths for each of the sample analytes; and identifying, in the database, a combination of concentrations of the sample analytes associated with absorption values that differ from the measured absorption values with least error.

In some embodiments, the method further comprises reporting the identified combination of concentrations as the concentrations of the sample analytes. The error may, for example, be the squared error or the absolute error. The method may further comprise interpolating among database entries and reporting interpolated concentrations as the concentrations of the sample analytes.

In another aspect, the invention relates to a system for determining concentrations in a sample containing a plurality of known analytes at unknown concentrations, where the sample analytes have different absorption spectra. In various embodiments, the system comprises a spectrophotometer, a database of absorbance values for multiple analytes each at multiple concentration levels and at multiple wavelengths, and a processor configured to (i) operate the spectrophotometer to measure radiation absorptions of the sample at a plurality of wavelengths of incident radiation, the plurality of wavelengths corresponding to peak absorption wavelengths for each of the sample analytes, and (ii) identify, in the database, a combination of concentrations of the sample analytes associated with absorption values that differ from the measured absorption values with least error.

The system may further comprise a display for reporting the identified combination of concentrations as the concentrations of the sample analytes and/or a network interface for reporting the identified combination of concentrations as the concentrations of the sample analytes. The error may be, for example, the squared or absolute error. In some embodiments, the processor is further configured to interpolate among database entries and report interpolated concentrations as the concentrations of the sample analytes.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The term “consists essentially of” means excluding other materials that contribute to function, unless otherwise defined herein. Nonetheless, such other materials may be present, collectively or individually, in trace amounts. The headings provided herein are for convenience only and are not intended to limit or interpret the scope or meaning of the claimed technology.

### BRIEF DESCRIPTION OF THE DRAWING

The foregoing discussion will be understood more readily from the following detailed description of the disclosed technology, when taken in conjunction with the single FIGURE of the drawing, which schematically illustrates a representative embodiment of a system in accordance with the present invention.

### DETAILED DESCRIPTION

In accordance with embodiments of the present invention, the absorbance of a mixed sample at multiple wavelengths is determined and the concentrations of the sample constituents deduced from the observed absorbances. Assuming the sample constituents are known, these wavelengths correspond to peak absorption wavelengths for the constituents. Rather than attempt to generate an analytical relationship among absorbance levels and constituent concentrations, a database of absorbance values for each wavelength, spanning the range of possible analyte concentrations, is employed instead. In general, the wavelengths utilized correspond to peak absorption wavelengths for each of the analytes.

Conceptually, this may be understood as follows. Suppose that a spectrophotometer is to be employed to analyze various samples each containing the same three analytes A, B, and C at unknown concentrations. (Of course, the invention is applicable to fewer (e.g., two) or more than three analytes, so long as their identities are known.) First, a calibration set is created. As shown in Table 1, this set is a list of detector responses for each analyte at each of three wavelengths; each of the wavelengths is absorbed most strongly by one of the analytes. The concentrations used for the calibration set span the concentrations that may be found in samples to be tested; in Table 1, these are 0, 0.2, 0.4, 0.6, 0.8, and 1, in arbitrary normalized units, i.e., a concentration of 1 does not necessarily correspond to 100%, but instead to the maximum concentration of the calibration range. In general, it is not necessary for all concentrations to be represented for each analyte so long as sufficient data exists for accurate calibration. In this example, six evenly spaced concentration levels are used. Alternatively, a linear or nonlinear equation may be used to generate the concentrations for each analyte.

The present approach is applicable both within and outside the linear region of the Beer-Lambert law—in the latter case, for example, when analyte concentrations are very high or the absorbance wavelength chosen does not include an absorbance peak for a given analyte. This is because the list of detector responses versus absorbance allows for any arbitrary shape rather than only a linear relationship between concentration and absorbance. Table 1 contains non-linear data and represents the general case. In the linear range, a multiple linear regression may be employed.

Labeling the analytes as Analyte A, Analyte B, and Analyte C, and the wavelengths tested as Wavelength 1, Wavelength 2, and Wavelength 3, the absorbances of the sample at each tested wavelength in this example are:

TABLE 1

| | | | |
|---|---|---|---|
| Concentration | Wavelength 1 | Wavelength 2 | Wavelength 3 |

Analyte A

| | | | |
|---|---|---|---|
| 0 | 0 | 0 | 0 |
| 0.2 | 0.2 | 0.1 | 0.05 |
| 0.4 | 0.4 | 0.2 | 0.1 |
| 0.6 | 0.6 | 0.3 | 0.15 |
| 0.8 | 0.8 | 0.4 | 0.2 |
| 1 | 1 | 0.5 | 0.25 |

Analyte B

| | | | |
|---|---|---|---|
| 0 | 0 | 0 | 0 |
| 0.2 | 0.2 | 0.2 | 0.1 |
| 0.4 | 0.3 | 0.4 | 0.4 |
| 0.6 | 0.4 | 0.6 | 0.5 |
| 0.8 | 0.6 | 0.8 | 0.6 |
| 1 | 0.8 | 1 | 0.7 |

Analyte C

| | | | |
|---|---|---|---|
| 0 | 0 | 0 | 0 |
| 0.2 | 0.05 | 0 | 0.2 |
| 0.4 | 0.1 | 0 | 0.4 |
| 0.6 | 0.15 | 0 | 0.6 |
| 0.8 | 0.2 | 0 | 0.8 |
| 1 | 0.25 | 0 | 1 |

Thus, Wavelength 1 is most strongly absorbed by Analyte A, Wavelength 2 is most strongly absorbed by Analyte B, and Wavelength 3 is most strongly absorbed by Analyte C.

The database for this example appears in Appendix 1 and is created as a table using the six concentration levels for each analyte shown in Table 1 (though, again, more or fewer than six levels may be used depending on the desired accuracy). Each row of the database corresponds to a different combination of concentrations for a total of 6³=216 rows. For each combination of concentrations in the Solution Matrix, the expected detector response (absorbance) is computed based on the calibration values, i.e., assuming that the concentration-dependent absorbance of each analyte in a mixture is the same as in a pure solution; these are shown as the Calculated Detector Response. Hence, each row of the table indexes a unique combination of analyte concentrations and the expected absorbances of each analyte in that combination.

Now suppose the absorbances of one particular sample at each tested wavelength are measured as follows:

| | |
|---|---|
| Wavelength 1 | 0.69 |
| Wavelength 2 | 0.5 |
| Wavelength 3 | 0.82 |

To estimate the unknown concentrations based on the above absorbance readings, the L1 (absolute) or L2 (squared) errors are computed; the squared errors are listed in Appendix 2. In particular, the error is computed, for each wavelength, as the difference between the measured absorbance and the corresponding Calculated Detector Response entry. For L2 error, this entry is squared, and each row of squared errors is summed. Thus, for the fourth row, the sum of squared errors is given as (0.69−0.15)²+(0.5−0)²+(0.82−0.60)²=0.590. The row having the minimum sum of squared errors across all tested wavelengths represents the best solution, i.e., the combination of concentrations that best accounts for the detector response. The lower the error, the better is the fit, and the precision of the solution depends on the number of table rows, i.e., the step size between concentration levels. It is possible to estimate more precise concentrations by interpolation. For example, in the case of a table with no equation, linear interpolation may be used between points as an estimate, and where an equation is employed it can be used to interpolate.

In some embodiments, the errors associated with different wavelengths are differently weighted in computing the error. For example, different wavelengths can have different degrees of analytical importance, or a wavelength may have a relatively lower absorbance, reducing the apparent error contribution, which is amplified to compensate. In these cases, the weighting factor may be greater than 1. In other cases, a wavelength may have high absorbance but low analytical importance in terms of analyte identification, and would therefore have a weighting factor of less than 1. For example, if Wavelength 1 were weighted by 2, the associated error becomes more important to the final result: 2×(0.69−0.15)²+(0.5−0)²+(0.82−0.60)²=0.882.

More precise concentration estimates may also be obtained using heuristics—i.e., any known relationships among analyte concentrations that might operate to preclude or favor certain combinations. Heuristics may also be used before calculation on the solution matrix to remove potential results known to be incorrect in order to reduce calculation time. For example, if the concentration of Analyte A can never be larger than that of Analyte B, then the size of the solution matrix can be reduced significantly.

For this example, the predicted (lowest squared error) solution occurs at index 51, corresponding to the following set of analyte concentrations:

| | |
|---|---|
| Analyte A | 0.20 |
| Analyte B | 0.40 |
| Analyte C | 0.40 |

Appendix 2 also shows the solutions sorted by the error magnitude.

It is possible to provide more than one solution to the user, who may use other methods (e.g., heuristics) to select the best one. For example, embodiments of the present invention may return the top 10 candidate solutions rather than the single best solution. The relative amount of error of the best solution at different wavelengths can also be returned. This error can assist in determining what, if any, interfering compounds may be present since the error at different wavelengths can in many cases correlate to the absorbance intensities expected from interfering compounds at the wavelengths measured.

While the invention may be employed in connection with any wavelength of analytically useful light, it is found to be particularly useful with mid-infrared (mid-IR) radiation (i.e., the wavelength range from 2 to 20 μm), where fundamental absorbance bands provide strong absorbance for different analytes at significantly different wavelengths. Mid-IR spectroscopy measures fundamental vibrational bands of various functional groups in a sample. These fundamental bands yield clean spectra ideal for determining sample composition and for the identification of samples using their unique mid-IR fingerprints.

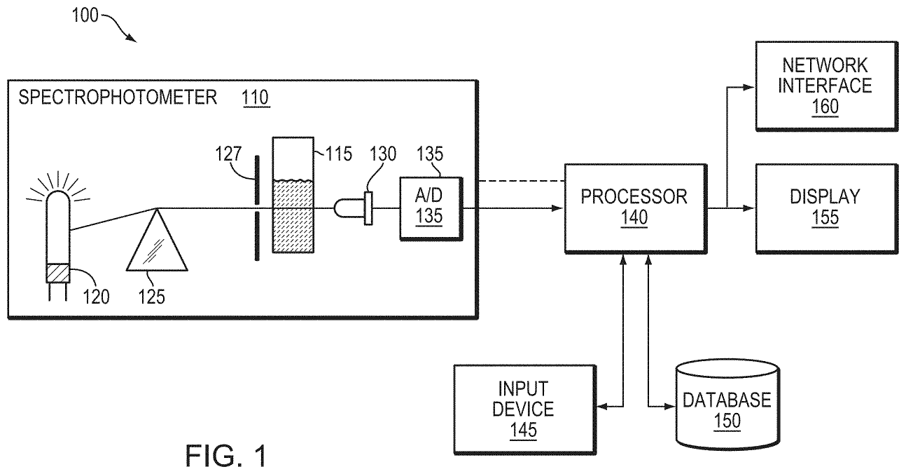

A representative system architecture 100 embodying the invention is illustrated in FIG. 1. A spectrophotometer 110 measures light intensity before and after introduction of a sample 115. The light is provided by a light source 120, which may emit at a single wavelength or, more commonly, is a broadband light source used with a bandpass filter to select a peak wavelength. Light from the source 120 which passes through a monochromator 125 containing a diffraction grating to produce an analytical spectrum, which itself passes through an aperture 127 (which may be adjustable) before reaching the sample 115. Relative movement between the light source 120 and the grating allows the light intensity at each wavelength to be measured, e.g., in a stepwise fashion. At each wavelength, the intensity of light emerging from the sample 115 is converted to an electrical signal by a photosensitive element 130 (e.g., a pyroelectric detector, bolometer, or mercuric cadmium telluride (MCT) detector) and the analog electrical signal is amplified and converted to digital form by an analog-to-digital (A/D) converter 135.

Alternative arrangements are possible. For example, gratings can suffer from temperature/shock effects, so light may be provided by a light source containing apertures or collimating optics with light passing through multiple fixed bandpass filters. Behind each bandpass filter a detector converts light intensity into electrical signal. In many cases, a multiple-element detector is used; this detector contains one or more filters above each detector or a continuously variable filter that varies its bandpass response across the filter.

Light may instead be provided by substantially monochromatic light sources, such as light-emitting diodes (LEDs). Each LED may contain collimating or guiding optics. All light sources are directed to a single detector, and each light source emits light in turn—i.e., only one light source is on at a time so that the generated detector signal is limited to a single wavelength. This can be achieved by pulsing power to the different emitters, or by use of a light chopper, which typically uses a motor to spin fan blades that momentarily block light from the light source(s).

A processor 140 controls the operation of the spectrophotometer 110 and receives the digital measurement signal from the A/D converter 135. The processor 140 also receives input from the user via an input device 145 (a keyboard, pointing device and display, touchscreen, barcode reader, etc.). For example, the user (or a label on the sample vial) may specify the analytes in the sample 115. A database 150 contains the calculated detector responses as set forth in Appendix 1 for numerous analytes. The database 150 also includes peak absorption wavelengths for these analytes. Based on the identified analytes in the sample 115, the processor 140 is programmed to query the database 150 and retrieve, into volatile or other working memory, the columns of the calculated detector responses and peak wavelengths corresponding to the identified analytes. The processor 140 is programmed to control the spectrophotometer 110 to obtain measurements at the peak wavelengths, to compute the error (e.g., L1 or L2) against the retrieved calculated detector responses, and to identify the relative analyte concentrations having the lowest error. The identified relative concentrations may be presented on a display 155 (which may be combined with the input device 145 as a touchscreen) and/or communicated via the Internet or other network over a conventional wired or wireless network interface 160.

The database 150 may be implemented as a table (e.g., a spreadsheet), or as a flat-file or relational database, and stored in a nonvolatile storage medium such as a hard disk, optical drive or Flash memory. In general, the database 150 may be constructed, populated, and queried on a computing platform, i.e., the system 100 or other platform. More generally, the system 100 may be a unitary device build around the spectrophotometer 110 or, alternatively, the processor 140, input device 145, display 155, and network interface spectrophotometer 110 may be realized on a desktop or laptop computer, or in a smart phone, tablet, or other mobile device. In the latter case, the device may control and receive data wirelessly from the spectrophotometer 110, and the database 150 may be maintained on the device or on a server accessed by the device, e.g., via the network interface 160 and the Internet. In this way, the database 150 may be centrally maintained and updated, and made available (e.g., on a subscription basis) to numerous “client” devices.

Suitable computing platforms typically include a variety of computer-readable media that can form part of the system memory and be read by the processing unit. By way of example, and not limitation, computer-readable media may comprise computer storage media and communication media. The system memory may include computer storage media in the form of volatile and/or nonvolatile memory such as read only memory (ROM) and random access memory (RAM). A basic input/output system (BIOS), containing the basic routines that help to transfer information between elements, such as during start-up, is typically stored in ROM. RAM typically contains data and/or program modules that are immediately accessible to and/or presently being operated on by processing unit. The data or program modules may include an operating system, application programs, other program modules, and program data. The operating system may be or include a variety of operating systems such as Microsoft WINDOWS operating system, the Unix operating system, the Linux operating system, the Xenix operating system, the IBM AIX operating system, the Hewlett Packard UX operating system, the Novell NETWARE operating system, the Sun Microsystems SOLARIS operating system, the OS/2 operating system, the BeOS operating system, the MACINTOSH operating system, the APACHE operating system, an OPENSTEP operating system or another operating system of platform.

The processor 140 may be a general-purpose microprocessor, a microcontroller, a peripheral integrated circuit element, a CSIC (customer-specific integrated circuit), an ASIC (application-specific integrated circuit), a logic circuit, a digital signal processor, a programmable logic device such as an FPGA (field-programmable gate array), PLD (programmable logic device), PLA (programmable logic array), or any other device or arrangement of devices that is capable of implementing the steps of the processes of the invention.

APPENDIX 1
Database

| Solution Matrix | | | | Calculated Detector Responses | | |
|---|---|---|---|---|---|---|
| index | Analyte A | Analyte B | Analyte C | Wavelength 1 | Wavelength 2 | Wavelength 3 |
| 1 | 0.0 | 0.0 | 0.0 | 0.00 | 0.00 | 0.00 |
| 2 | 0.0 | 0.0 | 0.2 | 0.05 | 0.00 | 0.20 |
| 3 | 0.0 | 0.0 | 0.4 | 0.10 | 0.00 | 0.40 |
| 4 | 0.0 | 0.0 | 0.6 | 0.15 | 0.00 | 0.60 |
| 5 | 0.0 | 0.0 | 0.8 | 0.20 | 0.00 | 0.80 |
| 6 | 0.0 | 0.0 | 1.0 | 0.25 | 0.00 | 1.00 |
| 7 | 0.0 | 0.2 | 0.0 | 0.20 | 0.20 | 0.10 |
| 8 | 0.0 | 0.2 | 0.2 | 0.25 | 0.20 | 0.30 |
| 9 | 0.0 | 0.2 | 0.4 | 0.30 | 0.20 | 0.50 |
| 10 | 0.0 | 0.2 | 0.6 | 0.35 | 0.20 | 0.70 |
| 11 | 0.0 | 0.2 | 0.8 | 0.40 | 0.20 | 0.90 |
| 12 | 0.0 | 0.2 | 1.0 | 0.45 | 0.20 | 1.10 |
| 13 | 0.0 | 0.4 | 0.0 | 0.30 | 0.40 | 0.40 |
| 14 | 0.0 | 0.4 | 0.2 | 0.35 | 0.40 | 0.60 |
| 15 | 0.0 | 0.4 | 0.4 | 0.40 | 0.40 | 0.80 |
| 16 | 0.0 | 0.4 | 0.6 | 0.45 | 0.40 | 1.00 |
| 17 | 0.0 | 0.4 | 0.8 | 0.50 | 0.40 | 1.20 |
| 18 | 0.0 | 0.4 | 1.0 | 0.55 | 0.40 | 1.40 |
| 19 | 0.0 | 0.6 | 0.0 | 0.40 | 0.60 | 0.50 |
| 20 | 0.0 | 0.6 | 0.2 | 0.45 | 0.60 | 0.70 |
| 21 | 0.0 | 0.6 | 0.4 | 0.50 | 0.60 | 0.90 |
| 22 | 0.0 | 0.6 | 0.6 | 0.55 | 0.60 | 1.10 |
| 23 | 0.0 | 0.6 | 0.8 | 0.60 | 0.60 | 1.30 |
| 24 | 0.0 | 0.6 | 1.0 | 0.65 | 0.60 | 1.50 |
| 25 | 0.0 | 0.8 | 0.0 | 0.60 | 0.80 | 0.60 |
| 26 | 0.0 | 0.8 | 0.2 | 0.65 | 0.80 | 0.80 |
| 27 | 0.0 | 0.8 | 0.4 | 0.70 | 0.80 | 1.00 |
| 28 | 0.0 | 0.8 | 0.6 | 0.75 | 0.80 | 1.20 |
| 29 | 0.0 | 0.8 | 0.8 | 0.80 | 0.80 | 1.40 |
| 30 | 0.0 | 0.8 | 1.0 | 0.85 | 0.80 | 1.60 |
| 31 | 0.0 | 1.0 | 0.0 | 0.80 | 1.00 | 0.70 |
| 32 | 0.0 | 1.0 | 0.2 | 0.85 | 1.00 | 0.90 |
| 33 | 0.0 | 1.0 | 0.4 | 0.90 | 1.00 | 1.10 |
| 34 | 0.0 | 1.0 | 0.6 | 0.95 | 1.00 | 1.30 |
| 35 | 0.0 | 1.0 | 0.8 | 1.00 | 1.00 | 1.50 |
| 36 | 0.0 | 1.0 | 1.0 | 1.05 | 1.00 | 1.70 |
| 37 | 0.2 | 0.0 | 0.0 | 0.20 | 0.10 | 0.05 |
| 38 | 0.2 | 0.0 | 0.2 | 0.25 | 0.10 | 0.25 |
| 39 | 0.2 | 0.0 | 0.4 | 0.30 | 0.10 | 0.45 |
| 40 | 0.2 | 0.0 | 0.6 | 0.35 | 0.10 | 0.65 |
| 41 | 0.2 | 0.0 | 0.8 | 0.40 | 0.10 | 0.85 |
| 42 | 0.2 | 0.0 | 1.0 | 0.45 | 0.10 | 1.05 |
| 43 | 0.2 | 0.2 | 0.0 | 0.40 | 0.30 | 0.15 |
| 44 | 0.2 | 0.2 | 0.2 | 0.45 | 0.30 | 0.35 |
| 45 | 0.2 | 0.2 | 0.4 | 0.50 | 0.30 | 0.55 |
| 46 | 0.2 | 0.2 | 0.6 | 0.55 | 0.30 | 0.75 |
| 47 | 0.2 | 0.2 | 0.8 | 0.60 | 0.30 | 0.95 |
| 48 | 0.2 | 0.2 | 1.0 | 0.65 | 0.30 | 1.15 |
| 49 | 0.2 | 0.4 | 0.0 | 0.50 | 0.50 | 0.45 |
| 50 | 0.2 | 0.4 | 0.2 | 0.55 | 0.50 | 0.65 |
| 51 | 0.2 | 0.4 | 0.4 | 0.60 | 0.50 | 0.85 |
| 52 | 0.2 | 0.4 | 0.6 | 0.65 | 0.50 | 1.05 |
| 53 | 0.2 | 0.4 | 0.8 | 0.70 | 0.50 | 1.25 |
| 54 | 0.2 | 0.4 | 1.0 | 0.75 | 0.50 | 1.45 |
| 55 | 0.2 | 0.6 | 0.0 | 0.60 | 0.70 | 0.55 |
| 56 | 0.2 | 0.6 | 0.2 | 0.65 | 0.70 | 0.75 |
| 57 | 0.2 | 0.6 | 0.4 | 0.70 | 0.70 | 0.95 |
| 58 | 0.2 | 0.6 | 0.6 | 0.75 | 0.70 | 1.15 |
| 59 | 0.2 | 0.6 | 0.8 | 0.80 | 0.70 | 1.35 |
| 60 | 0.2 | 0.6 | 1.0 | 0.85 | 0.70 | 1.55 |
| 61 | 0.2 | 0.8 | 0.0 | 0.80 | 0.90 | 0.65 |
| 62 | 0.2 | 0.8 | 0.2 | 0.85 | 0.90 | 0.85 |
| 63 | 0.2 | 0.8 | 0.4 | 0.90 | 0.90 | 1.05 |
| 64 | 0.2 | 0.8 | 0.6 | 0.95 | 0.90 | 1.25 |
| 65 | 0.2 | 0.8 | 0.8 | 1.00 | 0.90 | 1.45 |
| 66 | 0.2 | 0.8 | 1.0 | 1.05 | 0.90 | 1.65 |
| 67 | 0.2 | 1.0 | 0.0 | 1.00 | 1.10 | 0.75 |
| 68 | 0.2 | 1.0 | 0.2 | 1.05 | 1.10 | 0.95 |
| 69 | 0.2 | 1.0 | 0.4 | 1.10 | 1.10 | 1.15 |
| 70 | 0.2 | 1.0 | 0.6 | 1.15 | 1.10 | 1.35 |
| 71 | 0.2 | 1.0 | 0.8 | 1.20 | 1.10 | 1.55 |
| 72 | 0.2 | 1.0 | 1.0 | 1.25 | 1.10 | 1.75 |
| 73 | 0.4 | 0.0 | 0.0 | 0.40 | 0.20 | 0.10 |
| 74 | 0.4 | 0.0 | 0.2 | 0.45 | 0.20 | 0.30 |
| 75 | 0.4 | 0.0 | 0.4 | 0.50 | 0.20 | 0.50 |
| 76 | 0.4 | 0.0 | 0.6 | 0.55 | 0.20 | 0.70 |
| 77 | 0.4 | 0.0 | 0.8 | 0.60 | 0.20 | 0.90 |
| 78 | 0.4 | 0.0 | 1.0 | 0.65 | 0.20 | 1.10 |
| 79 | 0.4 | 0.2 | 0.0 | 0.60 | 0.40 | 0.20 |
| 80 | 0.4 | 0.2 | 0.2 | 0.65 | 0.40 | 0.40 |
| 81 | 0.4 | 0.2 | 0.4 | 0.70 | 0.40 | 0.60 |
| 82 | 0.4 | 0.2 | 0.6 | 0.75 | 0.40 | 0.80 |
| 83 | 0.4 | 0.2 | 0.8 | 0.80 | 0.40 | 1.00 |
| 84 | 0.4 | 0.2 | 1.0 | 0.85 | 0.40 | 1.20 |
| 85 | 0.4 | 0.4 | 0.0 | 0.70 | 0.60 | 0.50 |
| 86 | 0.4 | 0.4 | 0.2 | 0.75 | 0.60 | 0.70 |
| 87 | 0.4 | 0.4 | 0.4 | 0.80 | 0.60 | 0.90 |
| 88 | 0.4 | 0.4 | 0.6 | 0.85 | 0.60 | 1.10 |
| 89 | 0.4 | 0.4 | 0.8 | 0.90 | 0.60 | 1.30 |
| 90 | 0.4 | 0.4 | 1.0 | 0.95 | 0.60 | 1.50 |
| 91 | 0.4 | 0.6 | 0.0 | 0.80 | 0.80 | 0.60 |
| 92 | 0.4 | 0.6 | 0.2 | 0.85 | 0.80 | 0.80 |
| 93 | 0.4 | 0.6 | 0.4 | 0.90 | 0.80 | 1.00 |
| 94 | 0.4 | 0.6 | 0.6 | 0.95 | 0.80 | 1.20 |
| 95 | 0.4 | 0.6 | 0.8 | 1.00 | 0.80 | 1.40 |
| 96 | 0.4 | 0.6 | 1.0 | 1.05 | 0.80 | 1.60 |
| 97 | 0.4 | 0.8 | 0.0 | 1.00 | 1.00 | 0.70 |
| 98 | 0.4 | 0.8 | 0.2 | 1.05 | 1.00 | 0.90 |
| 99 | 0.4 | 0.8 | 0.4 | 1.10 | 1.00 | 1.10 |
| 100 | 0.4 | 0.8 | 0.6 | 1.15 | 1.00 | 1.30 |
| 101 | 0.4 | 0.8 | 0.8 | 1.20 | 1.00 | 1.50 |
| 102 | 0.4 | 0.8 | 1.0 | 1.25 | 1.00 | 1.70 |
| 103 | 0.4 | 1.0 | 0.0 | 1.20 | 1.20 | 0.80 |
| 104 | 0.4 | 1.0 | 0.2 | 1.25 | 1.20 | 1.00 |
| 105 | 0.4 | 1.0 | 0.4 | 1.30 | 1.20 | 1.20 |
| 106 | 0.4 | 1.0 | 0.6 | 1.35 | 1.20 | 1.40 |
| 107 | 0.4 | 1.0 | 0.8 | 1.40 | 1.20 | 1.60 |
| 108 | 0.4 | 1.0 | 1.0 | 1.45 | 1.20 | 1.80 |
| 109 | 0.6 | 0.0 | 0.0 | 0.60 | 0.30 | 0.15 |
| 110 | 0.6 | 0.0 | 0.2 | 0.65 | 0.30 | 0.35 |
| 111 | 0.6 | 0.0 | 0.4 | 0.70 | 0.30 | 0.55 |
| 112 | 0.6 | 0.0 | 0.6 | 0.75 | 0.30 | 0.75 |
| 113 | 0.6 | 0.0 | 0.8 | 0.80 | 0.30 | 0.95 |
| 114 | 0.6 | 0.0 | 1.0 | 0.85 | 0.30 | 1.15 |
| 115 | 0.6 | 0.2 | 0.0 | 0.80 | 0.50 | 0.25 |
| 116 | 0.6 | 0.2 | 0.2 | 0.85 | 0.50 | 0.45 |
| 117 | 0.6 | 0.2 | 0.4 | 0.90 | 0.50 | 0.65 |
| 118 | 0.6 | 0.2 | 0.6 | 0.95 | 0.50 | 0.85 |
| 119 | 0.6 | 0.2 | 0.8 | 1.00 | 0.50 | 1.05 |
| 120 | 0.6 | 0.2 | 1.0 | 1.05 | 0.50 | 1.25 |
| 121 | 0.6 | 0.4 | 0.0 | 0.90 | 0.70 | 0.55 |
| 122 | 0.6 | 0.4 | 0.2 | 0.95 | 0.70 | 0.75 |
| 123 | 0.6 | 0.4 | 0.4 | 1.00 | 0.70 | 0.95 |
| 124 | 0.6 | 0.4 | 0.6 | 1.05 | 0.70 | 1.15 |
| 125 | 0.6 | 0.4 | 0.8 | 1.10 | 0.70 | 1.35 |
| 126 | 0.6 | 0.4 | 1.0 | 1.15 | 0.70 | 1.55 |
| 127 | 0.6 | 0.6 | 0.0 | 1.00 | 0.90 | 0.65 |
| 128 | 0.6 | 0.6 | 0.2 | 1.05 | 0.90 | 0.85 |
| 129 | 0.6 | 0.6 | 0.4 | 1.10 | 0.90 | 1.05 |
| 130 | 0.6 | 0.6 | 0.6 | 1.15 | 0.90 | 1.25 |
| 131 | 0.6 | 0.6 | 0.8 | 1.20 | 0.90 | 1.45 |
| 132 | 0.6 | 0.6 | 1.0 | 1.25 | 0.90 | 1.65 |
| 133 | 0.6 | 0.8 | 0.0 | 1.20 | 1.10 | 0.75 |
| 134 | 0.6 | 0.8 | 0.2 | 1.25 | 1.10 | 0.95 |
| 135 | 0.6 | 0.8 | 0.4 | 1.30 | 1.10 | 1.15 |
| 136 | 0.6 | 0.8 | 0.6 | 1.35 | 1.10 | 1.35 |
| 137 | 0.6 | 0.8 | 0.8 | 1.40 | 1.10 | 1.55 |
| 138 | 0.6 | 0.8 | 1.0 | 1.45 | 1.10 | 1.75 |
| 139 | 0.6 | 1.0 | 0.0 | 1.40 | 1.30 | 0.85 |
| 140 | 0.6 | 1.0 | 0.2 | 1.45 | 1.30 | 1.05 |
| 141 | 0.6 | 1.0 | 0.4 | 1.50 | 1.30 | 1.25 |
| 142 | 0.6 | 1.0 | 0.6 | 1.55 | 1.30 | 1.45 |
| 143 | 0.6 | 1.0 | 0.8 | 1.60 | 1.30 | 1.65 |
| 144 | 0.6 | 1.0 | 1.0 | 1.65 | 1.30 | 1.85 |
| 145 | 0.8 | 0.0 | 0.0 | 0.80 | 0.40 | 0.20 |
| 146 | 0.8 | 0.0 | 0.2 | 0.85 | 0.40 | 0.40 |
| 147 | 0.8 | 0.0 | 0.4 | 0.90 | 0.40 | 0.60 |
| 148 | 0.8 | 0.0 | 0.6 | 0.95 | 0.40 | 0.80 |
| 149 | 0.8 | 0.0 | 0.8 | 1.00 | 0.40 | 1.00 |
| 150 | 0.8 | 0.0 | 1.0 | 1.05 | 0.40 | 1.20 |
| 151 | 0.8 | 0.2 | 0.0 | 1.00 | 0.60 | 0.30 |
| 152 | 0.8 | 0.2 | 0.2 | 1.05 | 0.60 | 0.50 |
| 153 | 0.8 | 0.2 | 0.4 | 1.10 | 0.60 | 0.70 |
| 154 | 0.8 | 0.2 | 0.6 | 1.15 | 0.60 | 0.90 |
| 155 | 0.8 | 0.2 | 0.8 | 1.20 | 0.60 | 1.10 |
| 156 | 0.8 | 0.2 | 1.0 | 1.25 | 0.60 | 1.30 |
| 157 | 0.8 | 0.4 | 0.0 | 1.10 | 0.80 | 0.60 |
| 158 | 0.8 | 0.4 | 0.2 | 1.15 | 0.80 | 0.80 |
| 159 | 0.8 | 0.4 | 0.4 | 1.20 | 0.80 | 1.00 |
| 160 | 0.8 | 0.4 | 0.6 | 1.25 | 0.80 | 1.20 |
| 161 | 0.8 | 0.4 | 0.8 | 1.30 | 0.80 | 1.40 |
| 162 | 0.8 | 0.4 | 1.0 | 1.35 | 0.80 | 1.60 |
| 163 | 0.8 | 0.6 | 0.0 | 1.20 | 1.00 | 0.70 |
| 164 | 0.8 | 0.6 | 0.2 | 1.25 | 1.00 | 0.90 |
| 165 | 0.8 | 0.6 | 0.4 | 1.30 | 1.00 | 1.10 |
| 166 | 0.8 | 0.6 | 0.6 | 1.35 | 1.00 | 1.30 |
| 167 | 0.8 | 0.6 | 0.8 | 1.40 | 1.00 | 1.50 |
| 168 | 0.8 | 0.6 | 1.0 | 1.45 | 1.00 | 1.70 |
| 169 | 0.8 | 0.8 | 0.0 | 1.40 | 1.20 | 0.80 |
| 170 | 0.8 | 0.8 | 0.2 | 1.45 | 1.20 | 1.00 |
| 171 | 0.8 | 0.8 | 0.4 | 1.50 | 1.20 | 1.20 |
| 172 | 0.8 | 0.8 | 0.6 | 1.55 | 1.20 | 1.40 |
| 173 | 0.8 | 0.8 | 0.8 | 1.60 | 1.20 | 1.60 |
| 174 | 0.8 | 0.8 | 1.0 | 1.65 | 1.20 | 1.80 |
| 175 | 0.8 | 1.0 | 0.0 | 1.60 | 1.40 | 0.90 |
| 176 | 0.8 | 1.0 | 0.2 | 1.65 | 1.40 | 1.10 |
| 177 | 0.8 | 1.0 | 0.4 | 1.70 | 1.40 | 1.30 |
| 178 | 0.8 | 1.0 | 0.6 | 1.75 | 1.40 | 1.50 |
| 179 | 0.8 | 1.0 | 0.8 | 1.80 | 1.40 | 1.70 |
| 180 | 0.8 | 1.0 | 1.0 | 1.85 | 1.40 | 1.90 |
| 181 | 1.0 | 0.0 | 0.0 | 1.00 | 0.50 | 0.25 |
| 182 | 1.0 | 0.0 | 0.2 | 1.05 | 0.50 | 0.45 |
| 183 | 1.0 | 0.0 | 0.4 | 1.10 | 0.50 | 0.65 |
| 184 | 1.0 | 0.0 | 0.6 | 1.15 | 0.50 | 0.85 |
| 185 | 1.0 | 0.0 | 0.8 | 1.20 | 0.50 | 1.05 |
| 186 | 1.0 | 0.0 | 1.0 | 1.25 | 0.50 | 1.25 |
| 187 | 1.0 | 0.2 | 0.0 | 1.20 | 0.70 | 0.35 |
| 188 | 1.0 | 0.2 | 0.2 | 1.25 | 0.70 | 0.55 |
| 189 | 1.0 | 0.2 | 0.4 | 1.30 | 0.70 | 0.75 |
| 190 | 1.0 | 0.2 | 0.6 | 1.35 | 0.70 | 0.95 |
| 191 | 1.0 | 0.2 | 0.8 | 1.40 | 0.70 | 1.15 |
| 192 | 1.0 | 0.2 | 1.0 | 1.45 | 0.70 | 1.35 |
| 193 | 1.0 | 0.4 | 0.0 | 1.30 | 0.90 | 0.65 |
| 194 | 1.0 | 0.4 | 0.2 | 1.35 | 0.90 | 0.85 |
| 195 | 1.0 | 0.4 | 0.4 | 1.40 | 0.90 | 1.05 |
| 196 | 1.0 | 0.4 | 0.6 | 1.45 | 0.90 | 1.25 |
| 197 | 1.0 | 0.4 | 0.8 | 1.50 | 0.90 | 1.45 |
| 198 | 1.0 | 0.4 | 1.0 | 1.55 | 0.90 | 1.65 |
| 199 | 1.0 | 0.6 | 0.0 | 1.40 | 1.10 | 0.75 |
| 200 | 1.0 | 0.6 | 0.2 | 1.45 | 1.10 | 0.95 |
| 201 | 1.0 | 0.6 | 0.4 | 1.50 | 1.10 | 1.15 |
| 202 | 1.0 | 0.6 | 0.6 | 1.55 | 1.10 | 1.35 |
| 203 | 1.0 | 0.6 | 0.8 | 1.60 | 1.10 | 1.55 |
| 204 | 1.0 | 0.6 | 1.0 | 1.65 | 1.10 | 1.75 |
| 205 | 1.0 | 0.8 | 0.0 | 1.60 | 1.30 | 0.85 |
| 206 | 1.0 | 0.8 | 0.2 | 1.65 | 1.30 | 1.05 |
| 207 | 1.0 | 0.8 | 0.4 | 1.70 | 1.30 | 1.25 |
| 208 | 1.0 | 0.8 | 0.6 | 1.75 | 1.30 | 1.45 |
| 209 | 1.0 | 0.8 | 0.8 | 1.80 | 1.30 | 1.65 |
| 210 | 1.0 | 0.8 | 1.0 | 1.85 | 1.30 | 1.85 |
| 211 | 1.0 | 1.0 | 0.0 | 1.80 | 1.50 | 0.95 |
| 212 | 1.0 | 1.0 | 0.2 | 1.85 | 1.50 | 1.15 |
| 213 | 1.0 | 1.0 | 0.4 | 1.90 | 1.50 | 1.35 |
| 214 | 1.0 | 1.0 | 0.6 | 1.95 | 1.50 | 1.55 |
| 215 | 1.0 | 1.0 | 0.8 | 2.00 | 1.50 | 1.75 |
| 216 | 1.0 | 1.0 | 1.0 | 2.05 | 1.50 | 1.95 |

APPENDIX 2
Analysis of Sample

| Squared Error | | | | Summed | Sorted Solutions | | | Summed |
|---|---|---|---|---|---|---|---|---|
| index | Err_WL1 | Err_WL2 | Err_WL3 | Error | Analyte A | Analyte B | Analyte C | Error |
| 1 | 0.48 | 0.25 | 0.67 | 1.399 | 0.20 | 0.40 | 0.40 | 0.009 |
| 2 | 0.41 | 0.25 | 0.38 | 1.044 | 0.40 | 0.20 | 0.60 | 0.014 |
| 3 | 0.35 | 0.25 | 0.18 | 0.775 | 0.40 | 0.40 | 0.20 | 0.028 |
| 4 | 0.29 | 0.25 | 0.05 | 0.590 | 0.40 | 0.40 | 0.40 | 0.029 |
| 5 | 0.24 | 0.25 | 0.00 | 0.491 | 0.20 | 0.60 | 0.20 | 0.047 |
| 6 | 0.19 | 0.25 | 0.03 | 0.476 | 0.20 | 0.40 | 0.20 | 0.048 |
| 7 | 0.24 | 0.09 | 0.52 | 0.849 | 0.60 | 0.00 | 0.60 | 0.049 |
| 8 | 0.19 | 0.09 | 0.27 | 0.554 | 0.00 | 0.60 | 0.40 | 0.053 |
| 9 | 0.15 | 0.09 | 0.10 | 0.345 | 0.40 | 0.20 | 0.80 | 0.055 |
| 10 | 0.12 | 0.09 | 0.01 | 0.220 | 0.20 | 0.40 | 0.60 | 0.055 |
| 11 | 0.08 | 0.09 | 0.01 | 0.181 | 0.20 | 0.60 | 0.40 | 0.057 |
| 12 | 0.06 | 0.09 | 0.08 | 0.226 | 0.40 | 0.20 | 0.40 | 0.059 |
| 13 | 0.15 | 0.01 | 0.18 | 0.339 | 0.20 | 0.20 | 0.60 | 0.064 |
| 14 | 0.12 | 0.01 | 0.05 | 0.174 | 0.20 | 0.20 | 0.80 | 0.065 |
| 15 | 0.08 | 0.01 | 0.00 | 0.094 | 0.60 | 0.20 | 0.60 | 0.069 |
| 16 | 0.06 | 0.01 | 0.03 | 0.100 | 0.60 | 0.00 | 0.80 | 0.069 |
| 17 | 0.04 | 0.01 | 0.14 | 0.191 | 0.60 | 0.20 | 0.40 | 0.073 |
| 18 | 0.02 | 0.01 | 0.34 | 0.366 | 0.80 | 0.00 | 0.60 | 0.078 |
| 19 | 0.08 | 0.01 | 0.10 | 0.197 | 0.00 | 0.60 | 0.20 | 0.082 |
| 20 | 0.06 | 0.01 | 0.01 | 0.082 | 0.00 | 0.80 | 0.20 | 0.092 |
| 21 | 0.04 | 0.01 | 0.01 | 0.053 | 0.00 | 0.40 | 0.40 | 0.094 |
| 22 | 0.02 | 0.01 | 0.08 | 0.108 | 0.00 | 0.40 | 0.60 | 0.100 |
| 23 | 0.01 | 0.01 | 0.23 | 0.249 | 0.80 | 0.00 | 0.40 | 0.103 |
| 24 | 0.00 | 0.01 | 0.46 | 0.474 | 0.40 | 0.00 | 0.80 | 0.105 |
| 25 | 0.01 | 0.09 | 0.05 | 0.147 | 0.00 | 0.60 | 0.60 | 0.108 |
| 26 | 0.00 | 0.09 | 0.00 | 0.092 | 0.60 | 0.40 | 0.20 | 0.113 |
| 27 | 0.00 | 0.09 | 0.03 | 0.123 | 0.40 | 0.40 | 0.00 | 0.113 |
| 28 | 0.00 | 0.09 | 0.14 | 0.238 | 0.60 | 0.00 | 0.40 | 0.113 |
| 29 | 0.01 | 0.09 | 0.34 | 0.439 | 0.40 | 0.40 | 0.60 | 0.114 |
| 30 | 0.03 | 0.09 | 0.61 | 0.724 | 0.40 | 0.60 | 0.20 | 0.116 |
| 31 | 0.01 | 0.25 | 0.01 | 0.277 | 0.20 | 0.60 | 0.00 | 0.121 |
| 32 | 0.03 | 0.25 | 0.01 | 0.282 | 0.00 | 0.80 | 0.40 | 0.123 |
| 33 | 0.04 | 0.25 | 0.08 | 0.373 | 0.40 | 0.00 | 0.60 | 0.124 |
| 34 | 0.07 | 0.25 | 0.23 | 0.548 | 0.80 | 0.00 | 0.80 | 0.139 |
| 35 | 0.10 | 0.25 | 0.46 | 0.809 | 0.00 | 0.80 | 0.00 | 0.147 |
| 36 | 0.13 | 0.25 | 0.77 | 1.154 | 0.20 | 0.20 | 0.40 | 0.149 |
| 37 | 0.24 | 0.16 | 0.59 | 0.993 | 0.60 | 0.20 | 0.80 | 0.149 |
| 38 | 0.19 | 0.16 | 0.32 | 0.679 | 0.40 | 0.60 | 0.00 | 0.151 |
| 39 | 0.15 | 0.16 | 0.14 | 0.449 | 0.20 | 0.20 | 1.00 | 0.151 |
| 40 | 0.12 | 0.16 | 0.03 | 0.305 | 0.20 | 0.60 | 0.60 | 0.153 |
| 41 | 0.08 | 0.16 | 0.00 | 0.245 | 0.60 | 0.40 | 0.40 | 0.153 |
| 42 | 0.06 | 0.16 | 0.05 | 0.271 | 0.60 | 0.40 | 0.00 | 0.157 |
| 43 | 0.08 | 0.04 | 0.45 | 0.573 | 0.60 | 0.20 | 0.20 | 0.163 |
| 44 | 0.06 | 0.04 | 0.22 | 0.319 | 0.40 | 0.60 | 0.40 | 0.167 |
| 45 | 0.04 | 0.04 | 0.07 | 0.149 | 0.40 | 0.00 | 1.00 | 0.170 |
| 46 | 0.02 | 0.04 | 0.00 | 0.064 | 0.20 | 0.40 | 0.00 | 0.173 |
| 47 | 0.01 | 0.04 | 0.02 | 0.065 | 0.00 | 0.40 | 0.20 | 0.174 |
| 48 | 0.00 | 0.04 | 0.11 | 0.151 | 0.60 | 0.00 | 1.00 | 0.175 |
| 49 | 0.04 | 0.00 | 0.14 | 0.173 | 0.40 | 0.20 | 1.00 | 0.180 |
| 50 | 0.02 | 0.00 | 0.03 | 0.048 | 0.00 | 0.20 | 0.80 | 0.181 |
| 51 | 0.01 | 0.00 | 0.00 | 0.009 | 0.20 | 0.40 | 0.80 | 0.185 |
| 52 | 0.00 | 0.00 | 0.05 | 0.055 | 0.20 | 0.80 | 0.20 | 0.187 |
| 53 | 0.00 | 0.00 | 0.18 | 0.185 | 0.40 | 0.20 | 0.20 | 0.188 |
| 54 | 0.00 | 0.00 | 0.40 | 0.401 | 0.00 | 0.40 | 0.80 | 0.191 |
| 55 | 0.01 | 0.04 | 0.07 | 0.121 | 0.80 | 0.20 | 0.40 | 0.193 |
| 56 | 0.00 | 0.04 | 0.00 | 0.047 | 0.00 | 0.60 | 0.00 | 0.197 |
| 57 | 0.00 | 0.04 | 0.02 | 0.057 | 1.00 | 0.00 | 0.40 | 0.197 |
| 58 | 0.00 | 0.04 | 0.11 | 0.153 | 0.20 | 0.80 | 0.00 | 0.201 |
| 59 | 0.01 | 0.04 | 0.28 | 0.333 | 0.80 | 0.00 | 0.20 | 0.212 |
| 60 | 0.03 | 0.04 | 0.53 | 0.599 | 1.00 | 0.00 | 0.60 | 0.213 |
| 61 | 0.01 | 0.16 | 0.03 | 0.201 | 0.00 | 0.20 | 0.60 | 0.220 |
| 62 | 0.03 | 0.16 | 0.00 | 0.187 | 0.00 | 0.20 | 1.00 | 0.226 |
| 63 | 0.04 | 0.16 | 0.05 | 0.257 | 0.80 | 0.20 | 0.60 | 0.228 |
| 64 | 0.07 | 0.16 | 0.18 | 0.413 | 0.40 | 0.00 | 0.40 | 0.229 |
| 65 | 0.10 | 0.16 | 0.40 | 0.653 | 0.00 | 0.80 | 0.60 | 0.238 |
| 66 | 0.13 | 0.16 | 0.69 | 0.979 | 0.80 | 0.20 | 0.20 | 0.242 |
| 67 | 0.10 | 0.36 | 0.00 | 0.461 | 0.20 | 0.00 | 0.80 | 0.245 |
| 68 | 0.13 | 0.36 | 0.02 | 0.507 | 0.00 | 0.60 | 0.80 | 0.249 |
| 69 | 0.17 | 0.36 | 0.11 | 0.637 | 0.20 | 0.80 | 0.40 | 0.257 |
| 70 | 0.21 | 0.36 | 0.28 | 0.853 | 0.60 | 0.00 | 0.20 | 0.263 |
| 71 | 0.26 | 0.36 | 0.53 | 1.153 | 1.00 | 0.00 | 0.20 | 0.267 |
| 72 | 0.31 | 0.36 | 0.86 | 1.539 | 0.20 | 0.00 | 1.00 | 0.271 |
| 73 | 0.08 | 0.09 | 0.52 | 0.693 | 0.00 | 1.00 | 0.00 | 0.277 |
| 74 | 0.06 | 0.09 | 0.27 | 0.418 | 0.60 | 0.40 | 0.60 | 0.279 |
| 75 | 0.04 | 0.09 | 0.10 | 0.229 | 0.00 | 1.00 | 0.20 | 0.282 |
| 76 | 0.02 | 0.09 | 0.01 | 0.124 | 0.80 | 0.00 | 1.00 | 0.284 |
| 77 | 0.01 | 0.09 | 0.01 | 0.105 | 0.40 | 0.40 | 0.80 | 0.285 |
| 78 | 0.00 | 0.09 | 0.08 | 0.170 | 0.60 | 0.60 | 0.00 | 0.285 |
| 79 | 0.01 | 0.01 | 0.38 | 0.403 | 0.60 | 0.60 | 0.20 | 0.291 |
| 80 | 0.00 | 0.01 | 0.18 | 0.188 | 0.80 | 0.40 | 0.20 | 0.302 |
| 81 | 0.00 | 0.01 | 0.05 | 0.059 | 0.40 | 0.60 | 0.60 | 0.302 |
| 82 | 0.00 | 0.01 | 0.00 | 0.014 | 0.20 | 0.00 | 0.60 | 0.305 |
| 83 | 0.01 | 0.01 | 0.03 | 0.055 | 0.80 | 0.40 | 0.00 | 0.307 |
| 84 | 0.03 | 0.01 | 0.14 | 0.180 | 1.00 | 0.00 | 0.80 | 0.313 |
| 85 | 0.00 | 0.01 | 0.10 | 0.113 | 0.60 | 0.20 | 1.00 | 0.315 |
| 86 | 0.00 | 0.01 | 0.01 | 0.028 | 0.20 | 0.20 | 0.20 | 0.319 |
| 87 | 0.01 | 0.01 | 0.01 | 0.029 | 0.20 | 0.60 | 0.80 | 0.333 |
| 88 | 0.03 | 0.01 | 0.08 | 0.114 | 0.60 | 0.20 | 0.00 | 0.337 |
| 89 | 0.04 | 0.01 | 0.23 | 0.285 | 0.00 | 0.40 | 0.00 | 0.339 |
| 90 | 0.07 | 0.01 | 0.46 | 0.540 | 0.00 | 0.20 | 0.40 | 0.345 |
| 91 | 0.01 | 0.09 | 0.05 | 0.151 | 0.80 | 0.20 | 0.80 | 0.349 |
| 92 | 0.03 | 0.09 | 0.00 | 0.116 | 0.40 | 0.80 | 0.00 | 0.361 |
| 93 | 0.04 | 0.09 | 0.03 | 0.167 | 0.00 | 0.40 | 1.00 | 0.366 |
| 94 | 0.07 | 0.09 | 0.14 | 0.302 | 0.00 | 1.00 | 0.40 | 0.373 |
| 95 | 0.10 | 0.09 | 0.34 | 0.523 | 0.80 | 0.20 | 0.00 | 0.377 |
| 96 | 0.13 | 0.09 | 0.61 | 0.828 | 0.60 | 0.60 | 0.40 | 0.381 |
| 97 | 0.10 | 0.25 | 0.01 | 0.361 | 0.80 | 0.40 | 0.40 | 0.383 |
| 98 | 0.13 | 0.25 | 0.01 | 0.386 | 0.40 | 0.80 | 0.20 | 0.386 |
| 99 | 0.17 | 0.25 | 0.08 | 0.497 | 0.20 | 0.40 | 1.00 | 0.401 |
| 100 | 0.21 | 0.25 | 0.23 | 0.692 | 0.40 | 0.20 | 0.00 | 0.403 |
| 101 | 0.26 | 0.25 | 0.46 | 0.973 | 0.80 | 0.00 | 0.00 | 0.407 |
| 102 | 0.31 | 0.25 | 0.77 | 1.338 | 0.20 | 0.80 | 0.60 | 0.413 |
| 103 | 0.26 | 0.49 | 0.00 | 0.751 | 1.00 | 0.20 | 0.40 | 0.417 |
| 104 | 0.31 | 0.49 | 0.03 | 0.836 | 0.40 | 0.00 | 0.20 | 0.418 |
| 105 | 0.37 | 0.49 | 0.14 | 1.007 | 1.00 | 0.00 | 0.00 | 0.421 |
| 106 | 0.44 | 0.49 | 0.34 | 1.262 | 1.00 | 0.20 | 0.20 | 0.427 |
| 107 | 0.50 | 0.49 | 0.61 | 1.603 | 0.00 | 0.80 | 0.80 | 0.439 |
| 108 | 0.58 | 0.49 | 0.96 | 2.028 | 0.20 | 0.00 | 0.40 | 0.449 |
| 109 | 0.01 | 0.04 | 0.45 | 0.497 | 0.20 | 1.00 | 0.00 | 0.461 |
| 110 | 0.00 | 0.04 | 0.22 | 0.263 | 0.00 | 0.60 | 1.00 | 0.474 |
| 111 | 0.00 | 0.04 | 0.07 | 0.113 | 0.00 | 0.00 | 1.00 | 0.476 |
| 112 | 0.00 | 0.04 | 0.00 | 0.049 | 0.60 | 0.40 | 0.80 | 0.489 |
| 113 | 0.01 | 0.04 | 0.02 | 0.069 | 0.00 | 0.00 | 0.80 | 0.491 |
| 114 | 0.03 | 0.04 | 0.11 | 0.175 | 1.00 | 0.20 | 0.60 | 0.493 |
| 115 | 0.01 | 0.00 | 0.32 | 0.337 | 0.40 | 0.80 | 0.40 | 0.497 |
| 116 | 0.03 | 0.00 | 0.14 | 0.163 | 0.60 | 0.00 | 0.00 | 0.497 |
| 117 | 0.04 | 0.00 | 0.03 | 0.073 | 1.00 | 0.00 | 1.00 | 0.499 |
| 118 | 0.07 | 0.00 | 0.00 | 0.069 | 0.20 | 1.00 | 0.20 | 0.507 |
| 119 | 0.10 | 0.00 | 0.05 | 0.149 | 1.00 | 0.20 | 0.00 | 0.521 |
| 120 | 0.13 | 0.00 | 0.18 | 0.315 | 0.40 | 0.60 | 0.80 | 0.523 |
| 121 | 0.04 | 0.04 | 0.07 | 0.157 | 0.80 | 0.60 | 0.00 | 0.525 |
| 122 | 0.07 | 0.04 | 0.00 | 0.113 | 0.40 | 0.40 | 1.00 | 0.540 |
| 123 | 0.10 | 0.04 | 0.02 | 0.153 | 0.00 | 1.00 | 0.60 | 0.548 |
| 124 | 0.13 | 0.04 | 0.11 | 0.279 | 0.80 | 0.40 | 0.60 | 0.548 |
| 125 | 0.17 | 0.04 | 0.28 | 0.489 | 0.00 | 0.20 | 0.20 | 0.554 |
| 126 | 0.21 | 0.04 | 0.53 | 0.785 | 0.80 | 0.20 | 1.00 | 0.554 |
| 127 | 0.10 | 0.16 | 0.03 | 0.285 | 0.60 | 0.60 | 0.60 | 0.557 |
| 128 | 0.13 | 0.16 | 0.00 | 0.291 | 1.00 | 0.40 | 0.00 | 0.561 |
| 129 | 0.17 | 0.16 | 0.05 | 0.381 | 0.80 | 0.60 | 0.20 | 0.570 |
| 130 | 0.21 | 0.16 | 0.18 | 0.557 | 0.20 | 0.20 | 0.00 | 0.573 |
| 131 | 0.26 | 0.16 | 0.40 | 0.817 | 0.00 | 0.00 | 0.60 | 0.590 |
| 132 | 0.31 | 0.16 | 0.69 | 1.163 | 1.00 | 0.40 | 0.20 | 0.597 |
| 133 | 0.26 | 0.36 | 0.00 | 0.625 | 0.20 | 0.60 | 1.00 | 0.599 |
| 134 | 0.31 | 0.36 | 0.02 | 0.691 | 0.60 | 0.80 | 0.00 | 0.625 |
| 135 | 0.37 | 0.36 | 0.11 | 0.841 | 0.20 | 1.00 | 0.40 | 0.637 |
| 136 | 0.44 | 0.36 | 0.28 | 1.077 | 0.20 | 0.80 | 0.80 | 0.653 |
| 137 | 0.50 | 0.36 | 0.53 | 1.397 | 0.20 | 0.80 | 0.80 | 0.653 |
| 138 | 0.58 | 0.36 | 0.86 | 1.803 | 0.20 | 0.00 | 0.20 | 0.679 |
| 139 | 0.50 | 0.64 | 0.00 | 1.145 | 0.60 | 0.80 | 0.20 | 0.691 |
| 140 | 0.58 | 0.64 | 0.05 | 1.271 | 0.40 | 0.80 | 0.60 | 0.692 |
| 141 | 0.66 | 0.64 | 0.18 | 1.481 | 0.40 | 0.00 | 0.00 | 0.693 |
| 142 | 0.74 | 0.64 | 0.40 | 1.777 | 0.80 | 0.60 | 0.40 | 0.701 |
| 143 | 0.83 | 0.64 | 0.69 | 2.157 | 1.00 | 0.40 | 0.40 | 0.717 |
| 144 | 0.92 | 0.64 | 1.06 | 2.623 | 0.00 | 0.80 | 1.00 | 0.724 |
| 145 | 0.01 | 0.01 | 0.38 | 0.407 | 0.40 | 1.00 | 0.00 | 0.751 |
| 146 | 0.03 | 0.01 | 0.18 | 0.212 | 0.00 | 0.00 | 0.40 | 0.775 |
| 147 | 0.04 | 0.01 | 0.05 | 0.103 | 0.60 | 0.40 | 1.00 | 0.785 |
| 148 | 0.07 | 0.01 | 0.00 | 0.078 | 0.80 | 0.40 | 0.80 | 0.799 |
| 149 | 0.10 | 0.01 | 0.03 | 0.139 | 0.00 | 1.00 | 0.80 | 0.809 |
| 150 | 0.13 | 0.01 | 0.14 | 0.284 | 0.60 | 0.60 | 0.80 | 0.817 |
| 151 | 0.10 | 0.01 | 0.27 | 0.377 | 0.40 | 0.60 | 1.00 | 0.828 |
| 152 | 0.13 | 0.01 | 0.10 | 0.242 | 0.40 | 1.00 | 0.20 | 0.836 |
| 153 | 0.17 | 0.01 | 0.01 | 0.193 | 0.60 | 0.80 | 0.40 | 0.841 |
| 154 | 0.21 | 0.01 | 0.01 | 0.228 | 0.00 | 0.20 | 0.00 | 0.849 |
| 155 | 0.26 | 0.01 | 0.08 | 0.349 | 0.20 | 1.00 | 0.60 | 0.853 |
| 156 | 0.31 | 0.01 | 0.23 | 0.554 | 1.00 | 0.60 | 0.00 | 0.869 |
| 157 | 0.17 | 0.09 | 0.05 | 0.307 | 1.00 | 0.20 | 1.00 | 0.899 |
| 158 | 0.21 | 0.09 | 0.00 | 0.302 | 0.80 | 0.60 | 0.60 | 0.916 |
| 159 | 0.26 | 0.09 | 0.03 | 0.383 | 1.00 | 0.40 | 0.60 | 0.923 |
| 160 | 0.31 | 0.09 | 0.14 | 0.548 | 1.00 | 0.60 | 0.20 | 0.955 |
| 161 | 0.37 | 0.09 | 0.34 | 0.799 | 0.40 | 0.80 | 0.80 | 0.973 |
| 162 | 0.44 | 0.09 | 0.61 | 1.134 | 0.20 | 0.80 | 1.00 | 0.979 |
| 163 | 0.26 | 0.25 | 0.01 | 0.525 | 0.20 | 0.00 | 0.00 | 0.993 |
| 164 | 0.31 | 0.25 | 0.01 | 0.570 | 0.80 | 0.80 | 0.00 | 0.995 |
| 165 | 0.37 | 0.25 | 0.08 | 0.701 | 0.40 | 1.00 | 0.40 | 1.007 |
| 166 | 0.44 | 0.25 | 0.23 | 0.916 | 0.00 | 0.00 | 0.20 | 1.044 |
| 167 | 0.50 | 0.25 | 0.46 | 1.217 | 0.60 | 0.80 | 0.60 | 1.077 |
| 168 | 0.58 | 0.25 | 0.77 | 1.602 | 0.80 | 0.80 | 0.20 | 1.100 |
| 169 | 0.50 | 0.49 | 0.00 | 0.995 | 1.00 | 0.60 | 0.40 | 1.125 |
| 170 | 0.58 | 0.49 | 0.03 | 1.100 | 0.80 | 0.40 | 1.00 | 1.134 |
| 171 | 0.66 | 0.49 | 0.14 | 1.291 | 0.60 | 1.00 | 0.00 | 1.145 |
| 172 | 0.74 | 0.49 | 0.34 | 1.566 | 0.20 | 1.00 | 0.80 | 1.153 |
| 173 | 0.83 | 0.49 | 0.61 | 1.927 | 0.00 | 1.00 | 1.00 | 1.154 |
| 174 | 0.92 | 0.49 | 0.96 | 2.372 | 0.60 | 0.60 | 1.00 | 1.163 |
| 175 | 0.83 | 0.81 | 0.01 | 1.645 | 1.00 | 0.40 | 0.80 | 1.213 |
| 176 | 0.92 | 0.81 | 0.08 | 1.810 | 0.80 | 0.60 | 0.80 | 1.217 |
| 177 | 1.02 | 0.81 | 0.23 | 2.061 | 0.40 | 1.00 | 0.60 | 1.262 |
| 178 | 1.12 | 0.81 | 0.46 | 2.396 | 0.60 | 1.00 | 0.20 | 1.271 |
| 179 | 1.23 | 0.81 | 0.77 | 2.817 | 0.80 | 0.80 | 0.40 | 1.291 |
| 180 | 1.35 | 0.81 | 1.17 | 3.322 | 0.40 | 0.80 | 1.00 | 1.338 |
| 181 | 0.10 | 0.00 | 0.32 | 0.421 | 1.00 | 0.60 | 0.60 | 1.381 |
| 182 | 0.13 | 0.00 | 0.14 | 0.267 | 0.60 | 0.80 | 0.80 | 1.397 |
| 183 | 0.17 | 0.00 | 0.03 | 0.197 | 0.00 | 0.00 | 0.00 | 1.399 |
| 184 | 0.21 | 0.00 | 0.00 | 0.213 | 1.00 | 0.80 | 0.00 | 1.469 |
| 185 | 0.26 | 0.00 | 0.05 | 0.313 | 0.60 | 1.00 | 0.40 | 1.481 |
| 186 | 0.31 | 0.00 | 0.18 | 0.499 | 0.20 | 1.00 | 1.00 | 1.539 |
| 187 | 0.26 | 0.04 | 0.22 | 0.521 | 0.80 | 0.80 | 0.60 | 1.566 |
| 188 | 0.31 | 0.04 | 0.07 | 0.427 | 1.00 | 0.40 | 1.00 | 1.589 |
| 189 | 0.37 | 0.04 | 0.00 | 0.417 | 0.80 | 0.60 | 1.00 | 1.602 |
| 190 | 0.44 | 0.04 | 0.02 | 0.493 | 0.40 | 1.00 | 0.80 | 1.603 |
| 191 | 0.50 | 0.04 | 0.11 | 0.653 | 1.00 | 0.80 | 0.20 | 1.615 |
| 192 | 0.58 | 0.04 | 0.28 | 0.899 | 0.80 | 1.00 | 0.00 | 1.645 |
| 193 | 0.37 | 0.16 | 0.03 | 0.561 | 1.00 | 0.60 | 0.80 | 1.721 |
| 194 | 0.44 | 0.16 | 0.00 | 0.597 | 0.60 | 1.00 | 0.60 | 1.777 |
| 195 | 0.50 | 0.16 | 0.05 | 0.717 | 0.60 | 0.80 | 1.00 | 1.803 |
| 196 | 0.58 | 0.16 | 0.18 | 0.923 | 0.80 | 1.00 | 0.20 | 1.810 |
| 197 | 0.66 | 0.16 | 0.40 | 1.213 | 1.00 | 0.80 | 0.40 | 1.845 |
| 198 | 0.74 | 0.16 | 0.69 | 1.589 | 0.80 | 0.80 | 0.80 | 1.927 |
| 199 | 0.50 | 0.36 | 0.00 | 0.869 | 0.40 | 1.00 | 1.00 | 2.028 |
| 200 | 0.58 | 0.36 | 0.02 | 0.955 | 0.80 | 1.00 | 0.40 | 2.061 |
| 201 | 0.66 | 0.36 | 0.11 | 1.125 | 1.00 | 0.60 | 1.00 | 2.147 |
| 202 | 0.74 | 0.36 | 0.28 | 1.381 | 0.60 | 1.00 | 0.80 | 2.157 |
| 203 | 0.83 | 0.36 | 0.53 | 1.721 | 1.00 | 0.80 | 0.60 | 2.161 |
| 204 | 0.92 | 0.36 | 0.86 | 2.147 | 1.00 | 1.00 | 0.00 | 2.249 |
| 205 | 0.83 | 0.64 | 0.00 | 1.469 | 0.80 | 0.80 | 1.00 | 2.372 |
| 206 | 0.92 | 0.64 | 0.05 | 1.615 | 0.80 | 1.00 | 0.60 | 2.396 |
| 207 | 1.02 | 0.64 | 0.18 | 1.845 | 1.00 | 1.00 | 0.20 | 2.455 |
| 208 | 1.12 | 0.64 | 0.40 | 2.161 | 1.00 | 0.80 | 0.80 | 2.561 |
| 209 | 1.23 | 0.64 | 0.69 | 2.561 | 0.60 | 1.00 | 1.00 | 2.623 |
| 210 | 1.35 | 0.64 | 1.06 | 3.047 | 1.00 | 1.00 | 0.40 | 2.745 |
| 211 | 1.23 | 1.00 | 0.02 | 2.249 | 0.80 | 1.00 | 0.80 | 2.817 |
| 212 | 1.35 | 1.00 | 0.11 | 2.455 | 1.00 | 0.80 | 1.00 | 3.047 |
| 213 | 1.46 | 1.00 | 0.28 | 2.745 | 1.00 | 1.00 | 0.60 | 3.121 |
| 214 | 1.59 | 1.00 | 0.53 | 3.121 | 0.80 | 1.00 | 1.00 | 3.322 |
| 215 | 1.72 | 1.00 | 0.86 | 3.581 | 1.00 | 1.00 | 0.80 | 3.581 |
| 216 | 1.85 | 1.00 | 1.28 | 4.127 | 1.00 | 1.00 | 1.00 | 4.127 |

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A method of determining concentrations in a sample containing a plurality of known analytes at unknown concentrations, the sample analytes having different absorption spectra, the method comprising the steps of:
a. providing a database of absorption values for multiple analytes each at multiple concentration levels and at multiple wavelengths;
b. measuring, with a spectrophotometer, radiation absorptions of the sample at a plurality of wavelengths of incident radiation, the plurality of wavelengths corresponding to peak absorption wavelengths for each of the sample analytes; and
c. identifying, in the database, a combination of concentrations of the sample analytes associated with absorption values that differ from the measured absorption values with least error.

2. The method of claim 1, further comprising the step of reporting the identified combination of concentrations as the concentrations of the sample analytes.

3. The method of claim 1, wherein the error is squared error.

4. The method of claim 1, wherein the error is absolute error.

5. The method of claim 1, further comprising the step of interpolating among database entries and reporting interpolated concentrations as the concentrations of the sample analytes.

6. The method of claim 1, further comprising the step of weighting differences between a measured absorption value and absorption values in the database for at least one of the wavelengths.

7. A system for determining concentrations in a sample containing a plurality of known analytes at unknown concentrations, the sample analytes having different absorption spectra, the system comprising:
a. a spectrophotometer;
b. a database of absorbance values for multiple analytes each at multiple concentration levels and at multiple wavelengths; and
c. a processor configured to (i) operate the spectrophotometer to measure radiation absorptions of the sample at a plurality of wavelengths of incident radiation, the plurality of wavelengths corresponding to peak absorption wavelengths for each of the sample analytes, and (ii) identify, in the database, a combination of concentrations of the sample analytes associated with absorption values that differ from the measured absorption values with least error.

8. The system of claim 6, further comprising a display for reporting the identified combination of concentrations as the concentrations of the sample analytes.

9. The system of claim 6, further comprising a network interface for reporting the identified combination of concentrations as the concentrations of the sample analytes.

10. The system of claim 6, wherein the error is squared error.

11. The system of claim 6, wherein the error is absolute error.

12. The system of claim 6, wherein the processor is further configured to interpolate among database entries and report interpolated concentrations as the concentrations of the sample analytes.

13. The system of claim 6, wherein the processor is further configured to weight differences between a measured absorption value and absorption values in the database for at least one of the wavelengths.
